(19) United States
(12) Patent Application Publication
(10) Pub. No.: US 2017/0079559 A1
(43) Pub. Date: Mar. 23, 2017

(54) METHOD AND SYSTEM FOR COLLECTION OF FOOT GEOMETRY DATA

(71) Applicant: The Foot Foundation, Aspen, CO (US)

(72) Inventor: Eric Ward, Aspen, CO (US)

(73) Assignee: The Foot Foundation, Aspen, CO (US)

(21) Appl. No.: 15/271,442

(22) Filed: Sep. 21, 2016

Related U.S. Application Data
(60) Provisional application No. 62/221,555, filed on Sep. 21, 2015.

(51) Int. Cl. A61B 5/107; A61B 5/00

(52) U.S. Cl. CPC A61B 5/1078; A61B 5/1079; A61B 5/1074; A61B 5/4595; A61B 5/4836

(57) ABSTRACT

A foot geometry data collection system captures geometric data of a subject's foot revealing deviations in Varus and Valgus throughout dorsiflexion and abduction range of motion. The system places the foot into a suspended state of load such that the musculature and bones of the foot interact to convey forces to the ankle and knee yet constrain the ankle and knee to remain aligned in a neutral configuration so as to reveal to any Varus or Valgus deviations.

The present application relates to and claims the benefit of priority to U.S. Provisional Patent Application No. 62/221,555 filed 21 Sep. 2015 which is hereby incorporated by reference in its entirety for all purposes as if My set forth herein.

### BACKGROUND OF THE INVENTION

Field of the Invention

Embodiments of the present invention relate, in general, to orthotic inserts and more particularly to a method and system for the collection of biomechanical data useful in the creation and use of orthotic inserts.

Relevant Background

Orthotic devices are designed to distribute the stress of weight-bearing areas of the foot to maximize comfort and minimize trauma to the sole of the foot. There are a variety of foot/shoe insert balance systems and custom formed orthotics. One of the most notable systems is described in U.S. Pat. Nos. 6,845,568 and 6,564,465 entitled “High Performance Foot Bed for Sports Equipment”. In these and other disclosures of the prior art an array of full foot wedges are used, of varying degree of angular displacement, to provide a user with the correct foot position in relation to its environment. Each attempts to place the foot in a neutral position and to align the structural components of the foot, ankle, knee and hip. This alignment is largely controlled by foot inclination. Foot inclination refers to the angle along the length of the foot as viewed from the toe to the heel relative to a horizontal plane. Inclination, fore, and aft variances in angle, is different from pronation or supination that are side to side angle variances.

Variances in inclination and pronation are typically measured relative to level ground or a similar frame of reference. Throughout history humans attempt to level the world and remove deformations that might make is trip or fall. In the form of, floors, buildings, stairs, sidewalks, roads etc., all create an environment that is substantially flat and level. Variations in inclination and pronation are important because almost without exception we walk, stand, or run on flat ground. “Normal deformities” of the foot are directly attributable to a repetitive interaction with a man-made world and often manifest themselves in the form of chronic pains, or actual injuries to many parts of the body.

During a normal walking gait, the outside of the heel makes initial contact with the ground. Soon after impact the mid-outside portion of the foot makes contact with the surface and the foot thereafter “rolls” inward, to a certain degree, to complete contact with the ground. In the case of a normal foot, this system can support body weight with little effort. The rolling in of the foot optimally distributes the forces of impact across the system of arches built within a “normal healthy foot”. This movement or rolling action is called “pronation,” and is the natural movement of the foot. Technically speaking, pronation is a tri-planar movement composed of subtalar eversion, ankle dorsiflexion, and forefoot abduction. Pronation is also critical to proper shock absorption as a person walks or runs. Key to understanding pronation is to consider what occurs as a result of a structural bias that exists within almost every foot.

As suggested above, a specific and measurable amount of pronation is required for the foot to function effectively, however damage and injury can occur during long term excessive pronation situations. When excessive or over pronation occurs, the arches flatten out more which in turn loads the foot system adversely. As a result, muscles engage to compensate for a lacking structural support. Exponential tension is loaded to the tendons and ligaments, and these forces are opposed and counterbalanced, all the way up the kinetic chain. This muscular tension is required to establish and sustain balance and musculature compensation of this sort enables an individual who over prorates balance. One of reasonable skill in the relevant art can readily appreciate that individuals subject to over pronation and/or supination fatigue rapidly as their muscles are constantly compensating for misaligned structural components.

Structurally speaking the foot is a spring loaded complex of very strong triangle shapes, much like a girder system that form a three sided pyramid. The foot is very complex device comprising a plurality of bones, muscles, ligaments and tendons and at its core the foot is made up of interdependent levers with certain segments made of substantially in-elastic, yet spring loaded, banding tissue.

FIG. 1 presents a right perspective view of the skeletal structure of a right foot. The orientation and relationship of bones such as the tibia 110, calcaneus 120, talus 130 and metatarsals 150 are shown in FIG. 1. This skeletal representation however, fails to illustrate the interdependence in the foot between the bones of the skeleton and the soft tissue that surrounds and fashions the bones into a supporting structure. FIGS. 2 A-E provide a simplified representation of a foot to illustrate the interdependence of the foot structure including the soft tissues that interact with each of the bones. In FIGS. 2A-E the bones are represented by straight solid lines while soft tissue is shown as curved lines.

At a high level four interdependent triangles make up the foot structure forming a pyramid. Viewing a foot from the top down and as shown in FIG. 2C, a triangle, 215, 220, 240 can be seen from big toe ball of foot, to the little toe ball, to the ankle and back to the big toe. FIG. 2B presents a similar triangle 215, 235, 220 from the big toe ball, to the heel, to the ankle, and back to the ball of the big toe. FIG. 2A illustrates a triangle, 220, 230, 210 wherein the vertices include the ankle, the little toe ball and the heel. Viewed from the bottom up and as shown in FIG. 2D the lower triangle 235, 250, 240 is formed from the big toe ball, to little toe ball, to the heel, and back to big toe ball. In each triangle there are lever sections by which bones 150 move in conjunction with other bones 120, 130, 140, and a band 205 section (aka connective or soft tissue). The bands 205 are flexibly inelastic meaning. Finally FIG. 2E reconciles FIGS. 2A-2D to illustrate the pyramid like structure 210, 215, 220, 230, 235, 240 that is formed by the combination of the bones and soft banding tissue of the foot.

The connective tissue allows the foot system to be pliable while not under a load while at the same time being geometrically reliable, predictable and stable and able to carry the weight of the body or more, once engaged with the ground. This reliability and predictability enables a supportive structure and any deviations that the structure may possess are measurable. Referring back to FIG. 2B the line 235 extending from the heel to the big toe ball is substantially formed with connective tissue or banding 205. As loads are placed on the foot, the heel and forefoot, including the big toe, are displaced away from each other extending the length of the band between the heel and the big toe ball.

The band between the heel and the big toe ball is not unlike the string of a bow and arrow. When the bow is at rest the string is tight and the string holds the arms of the bow, in this case the heel and big toe ball, into a bent position preventing them from spreading too far apart. The string (band) holds the ends of the foot from flattening out and provides constant tension to the bones in the foot. A similar scenario exists for each of the toes and the connective tissue stretching between the heel and the toes as well as between each toe and metatarsal. In the case of a normal healthy foot, the geometry of these structures work together. When all the banding sections of the structure are loaded, the geometry of the entire foot system is measurable. And as one might expect, if any of the banding sections are excessively relaxed the foot becomes a non-weight bearing structure and the geometry is not useful as to predict in what configuration it will function most effectively. Pronation is one measure of structural geometry of the foot and refers to an inward roll of the foot during a normal gait. As an individual walks the heel makes first contact with the ground followed by the outer portion of the foot. As a step continues the foot rolls away from the outer portion and toward to the big toe such that as the foot is lifting away from the ground at the completion of the step the big two is the last portion of the foot to make contact with the ground.

“Supination” is the opposite of pronation and refers to the outward roll of the foot during normal motion. A key to understanding supination is that supination is caused by specific muscular tension. Supination overcomes and balances opposing structural bias built into the foot structure. However, excessive supination (outward rolling), because the muscular nature of this movement, places a large strain on the soft tissue that stabilize the ankle, and can lead to the ankle rolling completely over, resulting in systemic failure, more commonly known as ankle sprains, and, in an extreme case, total lateral ligament rupture. If the foot cannot relax because of its natural biases, movement becomes challenging and inefficient. Thus proper alignment of the foot as it interacts with a level walking surface is critical. This is when a foot balancing device can neutralize the natural biases and provide a more efficient, and effective environment from which to work.

For many sports balance and posture are just as important as pronation and while many foot beds have tried to address pronation issues few, if any, have addressed how the device may impact balance. A typical approach is the introduction of an “arch support” where the arch of the foot gets filled with some sort of supportive material to stop the arch's collapse. This is the equivalent to pushing upwards on the structural integrity of a stone archway. From a purely structural point of view, such an upward force on the keystone would undermine the keystone and weaken the strength of the arch structure. This approach misses the simple fact that arches themselves are as “sell-supporting structures”. The foot is no different. While comprised of many components, structurally, the foot is as simple to break down, understand and measure as steel girders in a building. Mechanical and structural engineers support an arch through the use of abutments and buttresses. In each case, these components deter the displacement of the ends of the arch. If the abutment is raised, so is the arch but without undermining the structural integrity of the arch itself. In essence to raise an arch requires that the abutments themselves be raised. The key is to support the ends of the arch not the middle.

Reducing excessive pronation or supination, controlling inclination and supporting the foot's arch have been long known as a means by which to improve an individual's support structure. What has been problematic is a means by which to efficiently and accurately collect data by which such a support or orthotic can be made.

What is needed, therefore, is a reliable means by which to capture the interaction of the foot and the ground, in a way that mimics an individual in motion such as walking and running. These and other limitations of the prior art are addressed by one more embodiments of the present invention. Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### SUMMARY OF THE INVENTION

Use present invention relates to a method and system for the collection of pertinent biomechanical data used to make decisions on how to best support the human body through orthotics.

One embodiment of the present invention includes a system for collection of foot geometry data having a frame circumscribing a data collection region that defines a collection plane. A pliable membrane is positioned within the data collection region and within the collection plane. This membrane includes an upper surface and a lower surface. The system also includes a stabilizing arm coupled to the frame that extends away from the collection plane so as to position a lower portion of a leg and the lower surface of the user's foot to be in contact with the upper surface of the pliable membrane. A data collection device is positioned below the frame and in direct view of the lower surface of the pliable membrane configured to measure deflection of the pliable membrane away from the collection plane.

Other features of the foot geometry data collection system described above includes a lateral axis of rotation extending through the collection plane and through a juncture of the stabilizing arm and the frame such that the frame is configured to pivot about the lateral axis of rotation with respect to the stabilizing arm. The system is configured so that the lateral axis of rotation extends through the ankle of the user to place the knee and ankle in a neutral/aligned configuration.

Another feature of the system is a vertical axis of rotation extending through the collection plane and perpendicular to die lateral axis of rotation and wherein the fame is configured to pivot about the vertical axis of rotation. Moreover, the data collection device includes a machine capable of executing instructions embodied as software and a plurality of software portions and one of said software portions is configured to identify angular deviations of the foot from the lower surface of the pliable membrane as displaced by the foot as the foot, ankle and knee retain a neutral foot geometry. Another software portions is configured to identify a neutral foot geometry of the lower surface, of the pliable membrane as displaced by the foot with respect a line parallel to the collection plane as the frame is displaced about the lateral axis.

The frame of the present invention is configured to independently rotate about the lateral axis of rotation and the vertical axis of rotation and measure an angular displacement with respect to Varus and Valgus throughout the user's range of motion. Accordingly, the data collection device measures a longitudinal angular displacement of the foot with respect to the collection plane in a variety of ranges of motion.

The foot geometry data collection system also includes aperture guides configured to capture the foot of the user on the upper surface of the pliable membrane and maximize contact between the lower surface of the foot and the upper surface of the pliable membrane as the pliable membrane is displaced.

Another embodiment of the present invention is a method for collection foot geometry data. The method includes circumscribing a data collection region by a frame defining a collection plane and thereafter positioning a pliable membrane within the data collection region and within the collection plane. The pliable membrane includes an upper surface and a lower surface. The method continues by stabilizing a lower portion of a leg and a foot of a user using a stabilizing arm coupled to the frame such that a lower surface of the foot of the user is in contact with the upper surface of the pliable membrane. By doing so the ankle of the user is positioned such that a lateral axis of rotation extends through the ankle and a juncture of the stabilizing arm and the frame.

Displacing the pliable membrane away from the collection plane provides a surface by which the collection of foot geometry data by a data collection device positioned below the frame, and in direct view of the lower surface of the pliable membrane, can be accomplished. The collection of data includes measuring an angular deviation of the foot about the longitudinal axis (Varus and Valgus) with respect to the collection plane frame of reference.

Additional steps of the method described above include pivoting the stabilizing arm about the lateral axis of rotation, and responsive to pivoting the stabilizing arm collecting one or more displaced angular deviation measurements of the foot about the longitudinal axis with respect to the collection plane.

The method can also include extending a longitudinal axis of rotation through the collection plane and perpendicular to the lateral axis of rotation and pivoting the frame about the lateral axis of rotation with respect to the stabilizing arm to result in dorsiflexion of the foot so that additional data with respect to Varus or Valgus can be collected as the foot, ankle and knee remain in a neutral alignment.

In this example of the present invention, collecting data includes identifying angular deviations of the lower surface of the pliable membrane as displaced by the foot as the foot retains a neutral configuration. Rotating the frame can occur independently about the lateral axis of rotation and the vertical axis of rotation.

Collecting foot geometry data also includes measuring angular displacement the foot about the longitudinal axis under a plurality of displacements of the ankle about the lateral axis and angular displacement the foot about the longitudinal axis under a plurality of displacements of the ankle about the vertical axis.

The features and advantages described in this disclosure and in the following detailed description are not all-inclusive. Many additional features and advantages will be apparent to one of ordinary skill in the relevant art in view of the drawings, specification, and claims hereof. Moreover, it should be noted that the language used in the specification has been principally selected for readability and instructional purposes and may not have been selected to delineate or circumscribe the inventive subject matter; reference to the claims is necessary to determine such inventive subject matter.

### BRIEF DESCRIPTION OF THE DRAWINGS

The aforementioned and other features and objects of the present invention and the manner of attaining them will become more apparent, and the invention itself will be best understood, by reference to the following description of one or more embodiments taken in conjunction with the accompanying drawings, wherein:

FIG. 1 shows right perspective view of a skeletal structure of a right foot as would be known to one of reasonable skill;

FIGS. 2A-2E present a simplified rendition of the interaction of the skeletal bones and soft tissue musculature of the foot as would be known to one of reasonable skill;

FIG. 3 is a perspective view of a foot geometry data collection system according to one embodiment of the present invention;

FIGS. 4A-4C present a front view of a foot geometry data collection system according to one embodiment of the present invention showing various Valgus/Varus deviations correlated with one or more positions of dorsiflexion shown in FIGS. 5A-5C;

FIGS. 5A-5C present a side view of a foot geometry data collection system according to one embodiment of the present invention showing one or more positions of dorsiflexion;

FIG. 6 presents a top view of a foot-bed data collection system according to one embodiment of the present invention with the foot in various angular positions of abduction and adduction; and

FIG. 7 is a flowchart of one method embodiment for collecting foot-bed data according to the present invention.

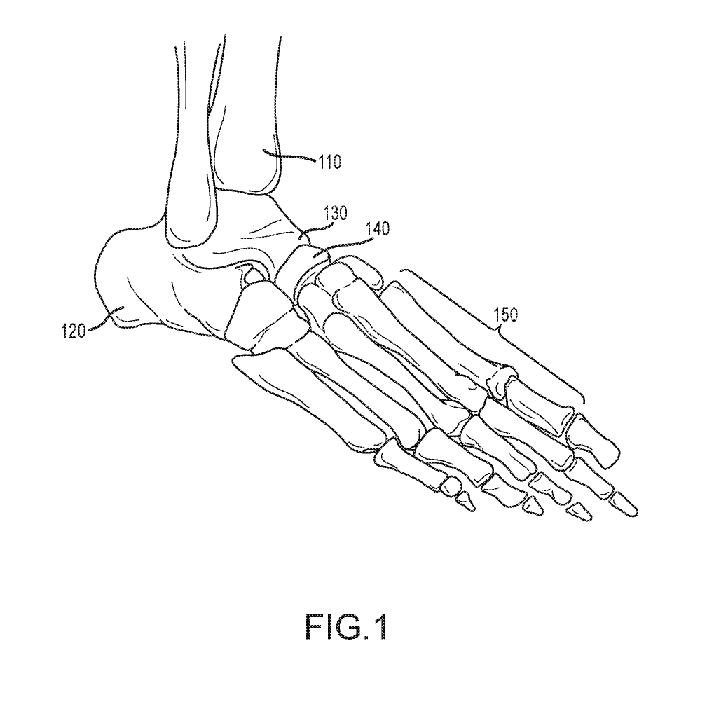

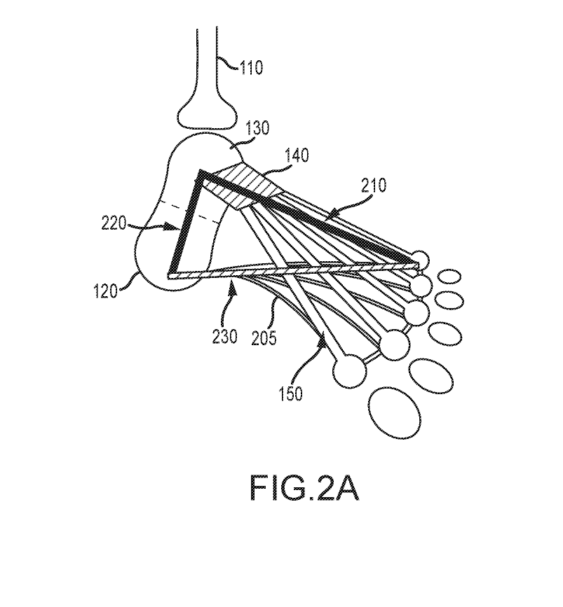

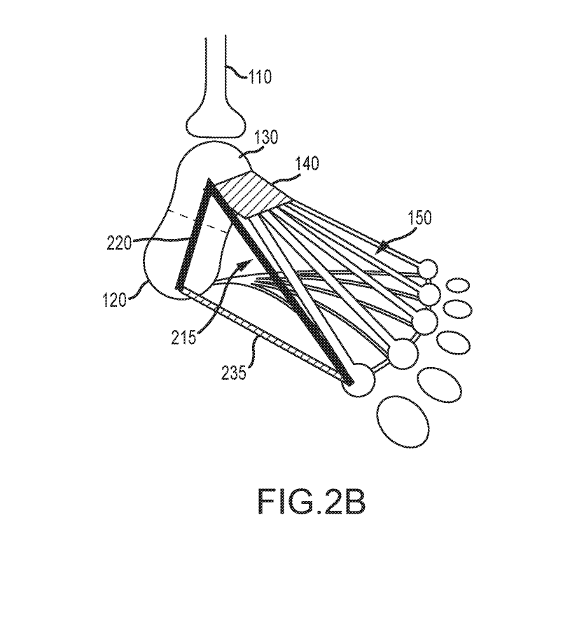

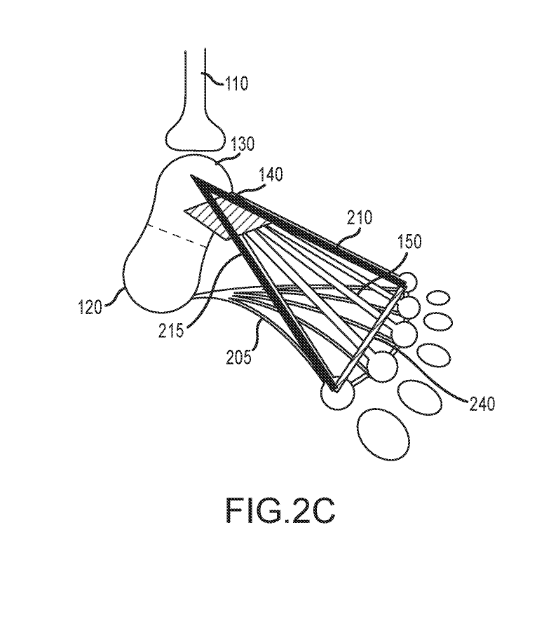

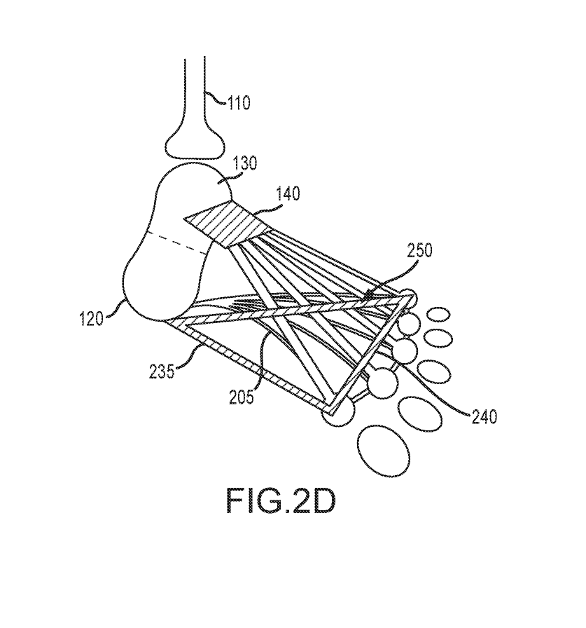

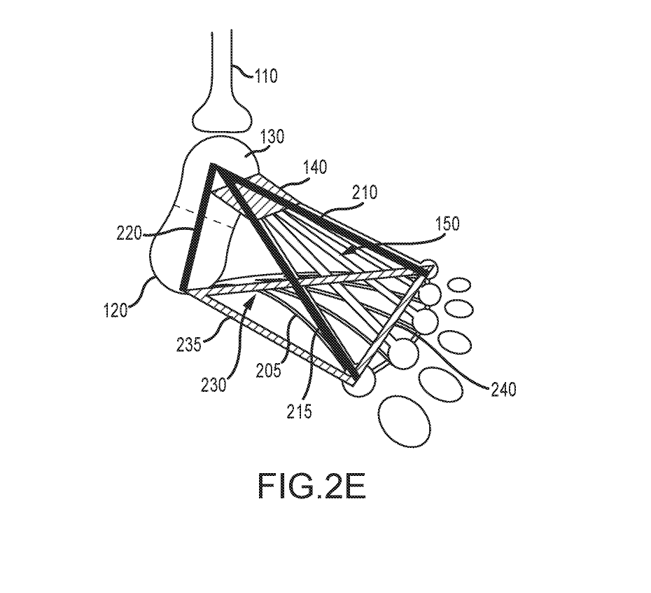

The Figures depict embodiments of the present invention for purposes of illustration only. One skilled in the art will readily recognize from the following discussion that alternative embodiments of the structures and methods illustrated herein may be employed without departing from the principles of the invention described herein.

### DESCRIPTION OF THE INVENTION

A deformable and articulable foot geometry data collection system captures pertinent geometric/biomechanical data of a subject's foot throughout the range of motion experienced during waking and/or running. Collected data is used to record and thereafter assess characteristics of the foot and surrounding musculature for production of a foot support or orthotic insert specific for that foot shape, geometry and intended functional use. The tool captures for future analysis unique features of an individual's foot while it experiences varying stages of load bearing environments yet while retaining the foot's neutral configuration.

As one of reasonable skill in the applicable art can appreciate, the shape of a foot and its interaction/alignment with the ankle, knee and hip (among other parts of the human anatomy) varies when subjected to different load conditions. The bones and surrounding musculature of the foot react differently when an individual is standing at an assembly line verses mining on a trail or walking down the street. Capturing this interaction and deviations of structural alignment is an objective of the present invention.

One embodiment of the present invention is to take a picture or digital image (scan) of the geometry of the foot with a 3-D scanning tool or similar topical reference technology. Such technology captures the topography of the foot once placed into the membrane of the molding de vice with respect to a reference plane and while under various loading conditions. The process described hereto is distinct from using foam boxes or plaster casts, or vacuum molding processes as it captures changes in the foot and its angular deviations while under changing loading conditions and with different angular dorsiflexion and abduction/adduction displacements while enabling the ankle, knee and hip to retain a neural configuration. Unlike foam boxes and plaster casts, the membrane of the present invention enables the foot structure to remain stable and in a neutral position so that deformities or bias of the foot in Valgus/Varus can be observed while the foot is under load.

Embodiments of the present invention are hereafter described in detail with reference to the accompanying Figures. Although the invention has been described and illustrated with a certain degree of particularity, it is understood that the present disclosure has been made only by way of example and that numerous changes in the combination and arrangement of parts can be resorted to by those skilled in the art without departing from the spirit and scope of the invention. The system described hereafter provides a means by which to capture the geometry of the foot with respect to its alignment with the ankle, knee and ultimately the hip. While the device and system introduced below and as described with reference to the accompanying drawings captures the geometry of the foot in its neutral configuration, one of reasonable skill in the relevant art will recognize that other configuration of such a device can be crafted to accomplish the same task. The focus of the invention is to place the foot into a suspended state of loading that engages the soft banding tissues associated with the skeleton of the foot while enabling the foot, ankle and knee to remain aligned. By doing so natural deviations or deformities in the combined foot skeleton/musculature can be recognized/measured and used to craft a suitable orthotic.

The following description with reference to the accompanying drawings is provided to assist in a comprehensive understanding of exemplary embodiments of the present invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. Also, descriptions of well-known functions and constructions are omitted for clarity and conciseness.

The terms and words used in the following description and claims, are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Like numbers refer to like elements throughout. In the Figures, the sizes of certain lines, fevers, components, elements or features may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Thus, for example, reference to “a component surface” includes reference to one or more of such surfaces.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is nor necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false for not present), A is false (or not present) and B is true (or present), and both A and B are true (or present).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

For the purpose of the present invention the following terms are defined.

Varus refers the inward angulation of the distal segment of a bone or joint while Valgus refers to the opposite direction. In the case of a foot a Varus angulation the foot tilts inward toward the inside or midline of the body, i.e. the big toe is higher than the little toe. In the present invention this movement is defined as movement about the y or longitudinal axis.

Abduction is the movement of a body part way from the midline while Adduction is movement toward the midline. With respect to feet, movement of the foot inward in a twisting movement rotating about the heel toward the midline of the body is adduction while movement aware from the midline is abduction. In the present invention this motion is defined as angular movement about the z or vertical axis.

Dorsiflexion is backward flexion of the foot as would be experienced by raising the toes of the foot toward the knee. Plantar flexion is extending the toes away from the knee or, more commonly known as, pointing the toes. The rotation of the foot about the ankle in dorsiflexion is, for the present invention, angular motion about the later or x axis.

It will be also understood that when an element is referred to as being “on,” “attached” to, “connected” to, “coupled” with, “contacting”, “mounted” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

One embodiment of the present invention uses a frame assembly with a flexible membrane material interposed/stretched between the interior section of the frame. The membrane, which is circumscribed by the frame, is pliable and flexible enough to allow the weight of the foot to deform the membrane into an exact replica of the foot as the foot presses into the membrane yet rigid enough to provide a representation of ground forces with which the foot would interact while the foot retains a neutral configuration. An individual sitting, standing, walking, or running can place their foot into this assembly and the elasticity of the material will load the banding (soft tissue) sections of the interdependent triangles described above, causing the foot to align into its optimal weight bearing position without being influenced by the surface of the ground. From this displacement the exact shape and orientation of the foot under load and undergoing a dynamic event can be captured digitally and measured against a frame of reference, or collection plane. Moreover, the geometry of the foot including deviations in Varus and Valgus can be captured as the foot extends through various ranges of dorsiflexion and abduction, all while the structure of the foot, ankle, knee and hip remain neutral.

The present invention captures the shape of both structure and banding components of a single foot while in dynamic equilibrium with a suspended loading force. Specifically, the fore foot Varus, or Valgus can be observed and measured as can the foot's movement in dorsiflexion. To do so the geometry (spatial position) of the three primary load bearing points of the foot, the heel, big toe ball and little toe ball, are compared and then extrapolated to identify the exact position and configuration of an orthotic device that would drive the foot structure to a neutral position upon the foot's interaction with the ground. As a result of changing the foot angle using an orthotic device, foot supportive structure is aligned including the ankle, knee, hip, pelvis, and spine. Movements will also be affected by the changes in foot position based on the initial readings taken with this new tool.

The device of the present invention allows the foot to retain its natural or neutral shape (orientation) and alignment during a dynamic event and while under a suspended load. Normally when a foot comes into contact with an immovable surface such as the ground, misalignment observed in the ankle, knee and hip joint is substantially due to excessive Varus or Valgus deformities. The degree of misalignment varies upon different degrees of dorsiflexion and adduction. The present invention enables the foot to undergo suspended loading as it would experience in a normal stride or gait yet allow the combined foot, ankle, knee and hip to remain aligned while presenting for observation deviations in Valgus and Varus. With the ankle, knee and hip aligned, a misaligned foot will show an angular displacement in the membrane with respect to the membrane frame of reference that can thereafter be used to craft a foot-bed to counter such misalignment.

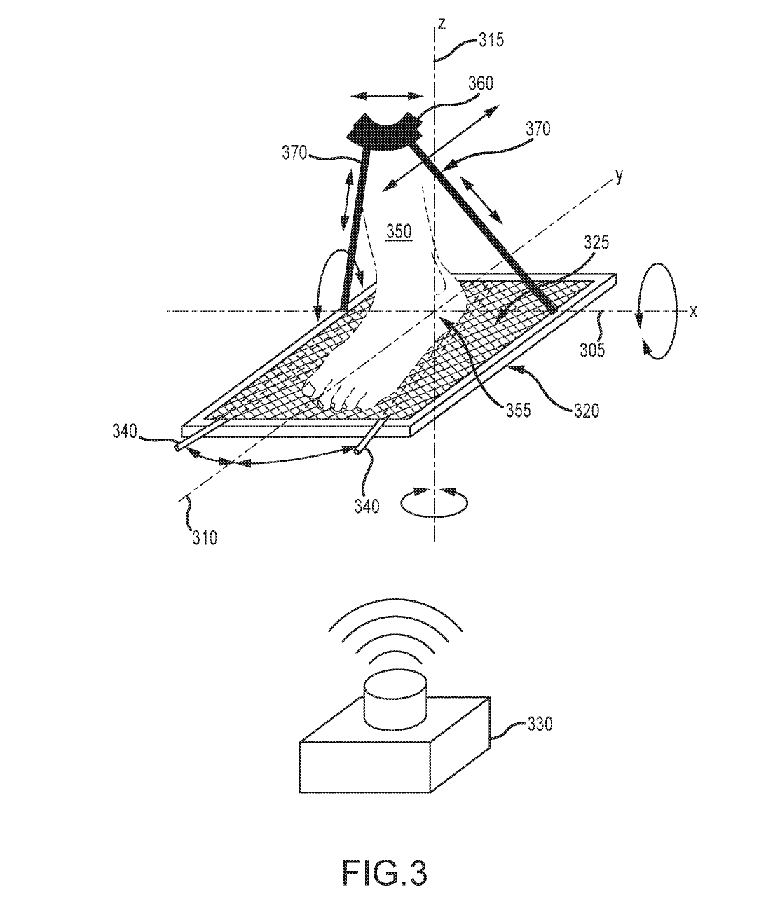

FIG. 3 is a perspective view of foot geometry data collection system, according to one embodiment of the present invention. A fame 320, or similar structure, circumscribes a pliable membrane 325 that defines a collection plane and local frame of reference. For the purposes of the present invention the collection plane is further defined as an x 305, y 310 plane comprised of lateral 305 and longitudinal 310 axes. The pliable membrane may be formed from a variety of elastic material that provides ample flexibility yet returns to its original shape with no permanent deformation. Moreover, the pliable membrane must exhibit substantially uniform elastic properties meaning that within the collection plane defined by the membrane, the longitudinal and lateral elasticity should be the substantially the same.

Coupled to the frame is a leg alignment or stabilization arm 360. As shown in FIG. 3, the stabilization arm 360 is composed of two supports 370 that extend away from the frame 320 in substantially a vertical 315 direction (in the z direction) and join together at a clasp or leg attachment device over the collection plane. The leg attachment device can be a strap, buckle, or hook and loop attachment means by which the lower portion of a user's leg can be secured to the system.

In one embodiment of the present invention, a user positions and secures their calf/tibia/leg 350 below the knee within the stabilization arm 360 such the bottom surface of their foot is in contact with, and displaces, the pliable membrane 325. A lateral axis 305, defined as the line running through the attachment point(s) of the stabilization arm to the frame and concurrent with the collection plane, traverses the ankle of the user such that, if the user would raise their toes toward the knee the ankle would pivot about the lateral axis. A longitudinal axis 310, perpendicular to the lateral axis 305 and concurrent with the collection plane, bisects the foot in FIG. 3 and traverses the ankle as well. Angular motion about the literal axis is known as dorsiflexion/plantar flexion. Angular motion about the longitudinal axis is Varus/Valgus while variation about the vertical axis is abduction/adduction of the foot. Note that the foot and leg of the user is fixed with respect to the longitudinal axis. Thus there is no angular motion or measurements about the longitudinal axis contemplated in the present invention other than what is the result of the displaced membrane. Therefore, the foot's displacement of the membrane will reveal to the data collection device degrees of Varus or Valgus deviation for different gradations of dorsiflexion and/or abduction.

The stabilisation arm 360 is movably coupled to the frame so as to enable the clasp or leg attachment device the ability to move forward or backward about the lateral axis. As the leg/knees moves forward resulting in dorsiflexion of the foot the supports 370 of the stabilization arm 360 may extend or retract independently. Similarly, if the collection plan is rotated about the vertical axis one stabilization arm may extend while the other retracts to maintain the heel of the foot in the proper position on the pliable membrane.

According to one embodiment of the present invention the collection system is configured to align the angle of the tibia/leg with respect to the bottom surface of the foot so that the displacement of the membrane relates to a normal tibia/foot angle as is present in an individual's normal movement patterns.

Another feature of the present invention are adjustable aperture rods 340 coupled to the frame and coexistent within the collection plane to enhance the ability of the membrane to capture the shape and angular deviations of the foot. As shown in FIG. 3, each aperture rod 340 is pivotally coupled with the rear portion of the frame 320 or the portion of the frame at which the heel of the user would be positioned for movement within the collection plane. Each aperture bar 340 extends along and to the side of the foot. By doing so the aperture bar will modify the displaceable surface area of the membrane to enable the shape of the sides of the foot to be effectively captured.

Below the pliable membrane and in full view of the collection region is one or more data collection devices 330. The data collection device 330, is, in one embodiment, a three-dimensional scanner, or the like, that can capture digitally a three dimensional shape of an object. The collection device also captures any deviation of the fame about the longitudinal or lateral axis to arrive at a comprehensive picture of the foot in a simulated dynamic loaded environment.

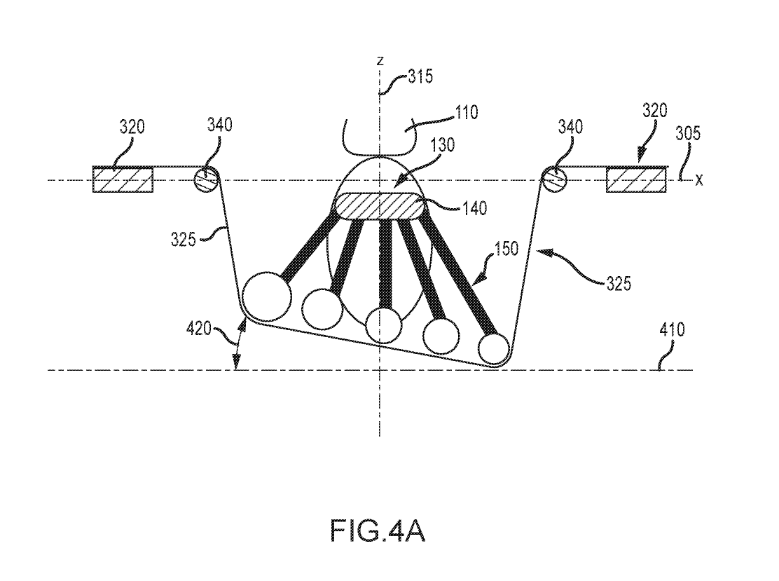

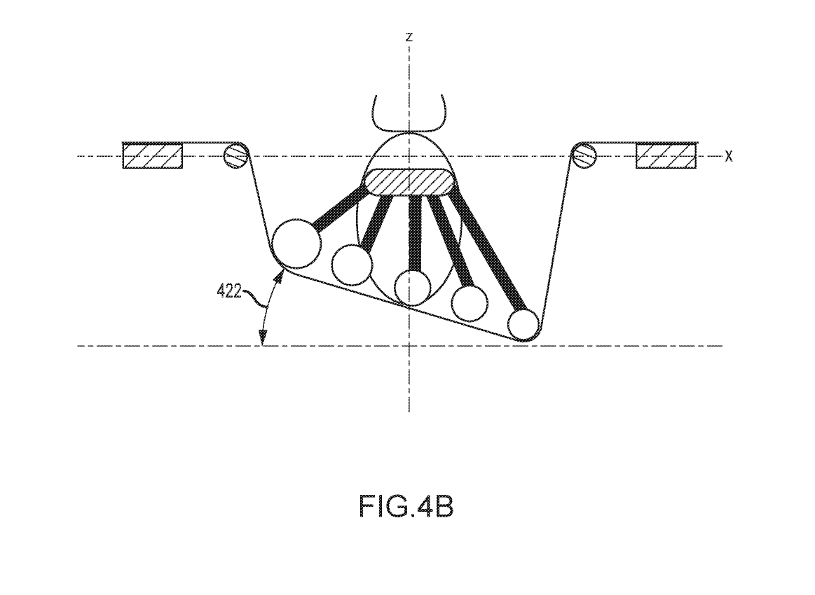

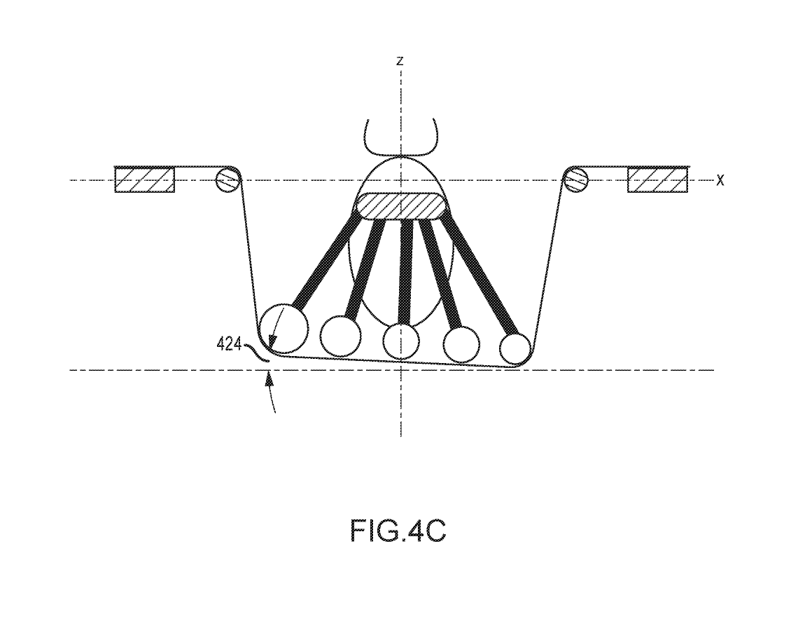

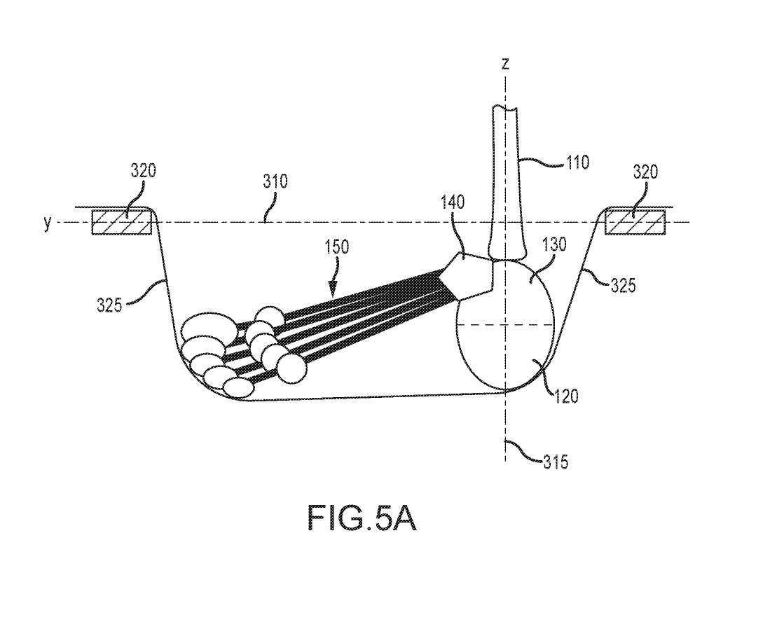

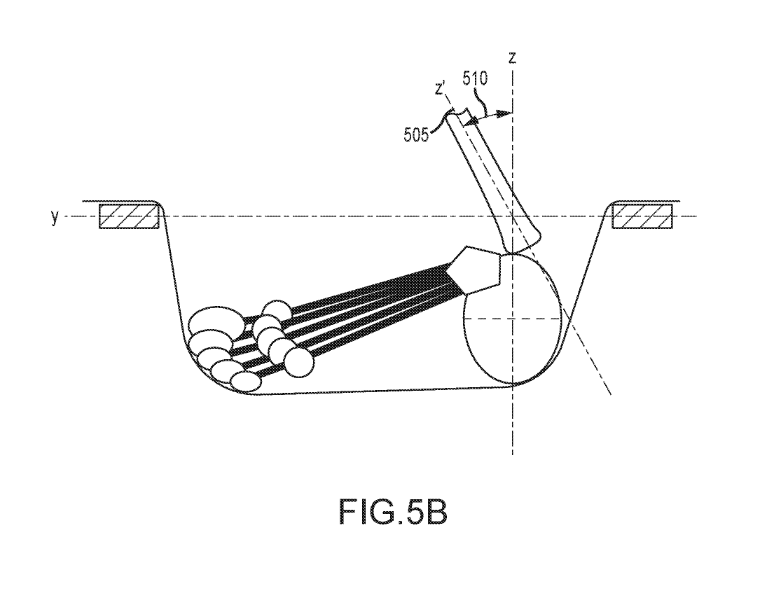

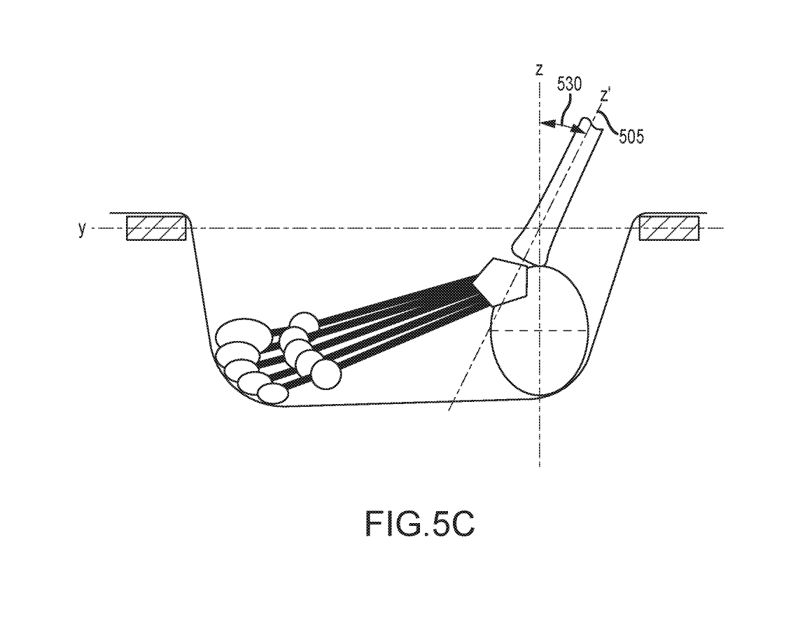

FIGS. 4A-4C each present a front view of the foot geometry data collection system, according to one embodiment of the present invention but with different degrees of Varus/Valgus. The vantage point of each FIG. 4 is from the front of the collection system looking toward the rear of the frame. In the rendition shown in FIG. 4A the foot of a user has displaced the pliable membrane 325 downward toward a ground surface frame of reference 410, in this instance the frame 320 and the membrane 325 reaction place the foot in a suspended state but with a neutral alignment of the ankle and knee. FIGS. 5A-5C present side views of the foot geometry data collection device. For the purpose of this discussion FIG. 4A corresponds to FIG. 5A, FIG. 4B corresponds to FIG. 5B and FIG. 4C corresponds to FIG. 5C.

As shown in FIGS. 4A and 5A, the ankle of the user is traversed by the lateral axis 305 with the heel displacing the membrane downward. The longitudinal axis, in FIG. 4A, bisects the foot and is directed in, and extending from, the surface of the page. The foot has displaced the membrane 325 under a representative suspension reaction force resulting in an angular variation 425 of the bottom surface of the foot. The foot in FIG. 4A illustrates an angular variation. 420 from the frame of reference 410 of the collection plane. One of reasonable skill in the relative art will recognize that by maintaining the relationship between the collection plane or plane of the displaced membrane and the leg, the ankle and knee remain aligned. Thus as the foot experiences a suspended load from displacing the membrane and the bones and banding tissues of the foot transfer the load to the ankle and knee, the foot retains it natural, neutral configuration. Assuming this is a left foot, the illustration shows a foot with a Varus deviation. While most individuals possess a certain degree of Varus, the deviation is not consistent from one person to another nor is it necessary consistent between an individual's two feet. Thus, in this illustration if the ground where tilted to match the Varus shown in FIG. 4A, the individual's ankle knee and hip would be aligned and the individual in a walking motion would need minimal musculature involvement to rectify any deviations in the bone structure. Said differently, the muscles would not have to work to keep the bones aligned since the bones are naturally aligned.

However, as this foot comes into contact with a real surface consistent with the ground, the foot will adopt the liar surface causing the ankle and knee to be misaligned. Muscles surrounding the ankle and knee compensate for the deviation. Overtime the muscle(s) can weaken or fail and even with strong muscles the endurance of one having an excessive Varus or Valgus deviation will be less than, that of a person, who is Varus/Valgus neutral or with a minimal deviation. Recognizing that this foot possesses a certain Varus deviation, a complementary foot-bed can be constructed matching the topology of the foot and eliminating the need for the muscular system of the foot to compensate for the misalignment. The challenge to create such a foot-bed is to bad the foot, ankle and knee while maintaining a neutral alignment to identify the true degree of Varus or Valgus.

One feature of the invention is to measure Varus/Valgus deviations under a suspended load while the ankle and knee remain aligned. Another feature of the invention is to place tire user in various states of dorsiflexion and/or abduction to gain additional information about the magnitude of Varus or Valgus and the position of the foot various. Significantly, as the foot is in a significant degree of dorsiflexion, that is as the foot is pushing off of the ground in a walking or running motion, the amount of Varus/Valgus deviation is maximized.

FIGS. 4B, 5B, 4C and 5C present different views of Varus/Valgus deviation as the foot is placed in various degrees of dorsiflexion. FIG. 4B shows that Varus 422 of the foot in question has increased as compared to FIG. 4A. Correspondingly, FIG. 5B represents that the leg of the user has been moved forward or toward the toes of the foot by certain angle 510. Thus the angle 510 between Z′ 505 and Y 310 is no longer 90 Degrees. Likewise. FIG. 4C presents a font view of the same foot but with a diminished degree of Varus 424. The corresponding side view of the foot geometry device of the present invention shows that the foot is in plantar flection in which the angle 530 between the Y 310 axis and Z′ 505 is greater than 90 degrees. Thus, the data, in this examples represents the increase in Varus of the foot as the leg and foot varies from a planter flection to dorsiflexion. Using this data, along with other information, an foot bed, foot insert or similar orthotic can be fashioned to maximize the ability of the knee and ankle to remain aligned throughput the range of motion.

Another feature of the present invention is that the collection system itself is, in one embodiment, suspended so as to enable the entire system freedom of rotation about the vertical and lateral axis. The rotational ability can also be independently constrained to isolate one or more particular measurements of interest including dorsiflexion, and abduction.

By doing so different conditions of use can also be examined. For example, while sitting and individual can extend their foot into its fully dorsiflexed position with a certain degree of abduction. This can be accomplished by rotating the foot at the ankle towards the knee and toward the medial line of the body. While in this situation, the foot is placed onto the membrane and then rotated. As the weight of leg and foot stretch the membrane, and, while remaking in this position the knee and ankle remain neutral but an image of the foot showing various degrees of Varus/Valgus can be collected. Of course each foot can be examined independently.

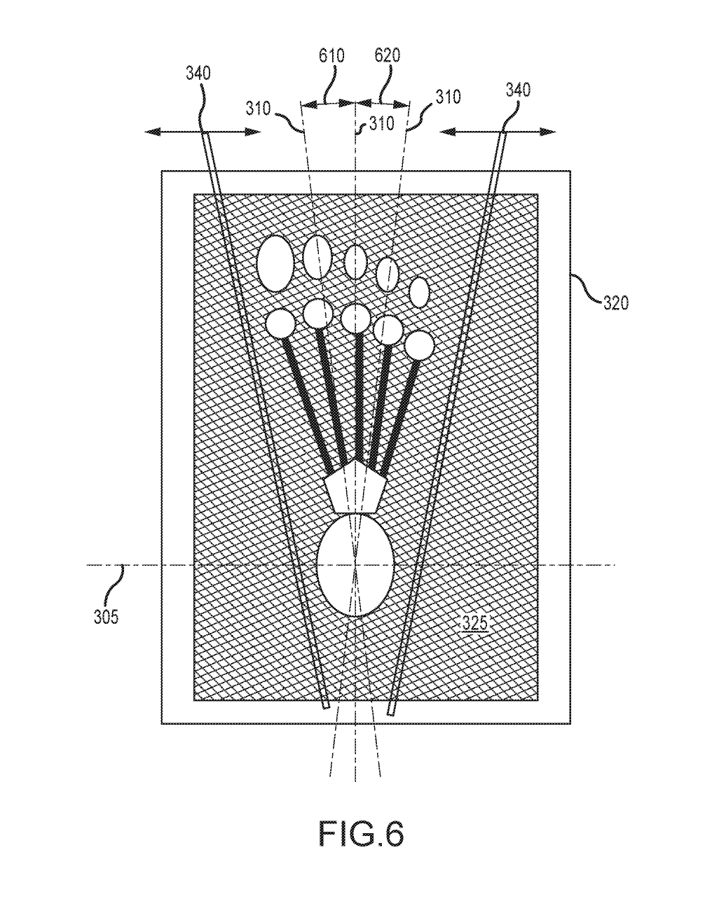

Accordingly, FIG. 6 shows a representative top view of the foot geometry system with the stabilization supports removed for clarity. In the rendition shown the heel of the foot 605 is traversed by the lateral axis 305 as it resides on the pliable membrane 325. As the foot is placed in different degrees of abduction or adduction various degrees of Varus (or Valgus as appropriate) can be determined. For example, if the foot as it resides in the frame and with the membrane displaced is rotated to the fell with respect to the knee in abduction by a certain angle 610 a varied amount of Varus or Valgus can be observed in the same manner as when the Varus or Valgus varied with respect to differences in dorsiflexion. Similarly, a deviation to right 620 or adduction may provide different changes in Varus or Valgus. As discussed above, at each of these variations about the vertical axis, the differences in Varus/Valgus can be observed and then combined with those found by varying dorsiflexion. The aperture bars 340 continue to constrain the collection region to optimize interaction between the foot and the pliable membrane.

One of reasonable skill in the relevant art will appreciate that several, different combinations of dorsiflexion, plantar flexion, abduction and adduction can be set to collect data, on the state of the foot's Varus or Valgus deviation. Ail of which is done with the foot under a suspended load so as to invoke the interaction of the soft tissue bands and rigid bones and while the ankle and knee are constrained to a neutral alignment.

A pressure sensitive membrane can also be used, in another embodiment to identify, and further calculate the rotational angle that most effectively and evenly distributes the pressure to the surface of the foot in the membrane without departing from the teachings and the scope of the present invention.

Use foot geometry collection system of the present invention captures information and useful measurements to ultimately construct a foot-bed so as to place an individual's foot into a neutral position while interacting with the ground. Turning back to FIG. 4A, the illustration shows that the left foot of this individual possesses a certain degree of Varus. Thus a complementary wedge placed in this person's left shoe would assist this individual to maintain an aligned structure through the ankle, knee and hip when walking or running on a flat surface.

Similar a measurement of fore/aft angular deviation about the lateral axis (dorsiflexion) may identify that for an individual to achieve a neutral position an orthotic must include a certain degree of inclination as well as compensation for Varus or Valgus. The present invention recognizes that the deviations with respect Varus and Valgus are not independent and that to achieve a functional and effective orthotic movement within the vertical and lateral axis of motion must be addressed.

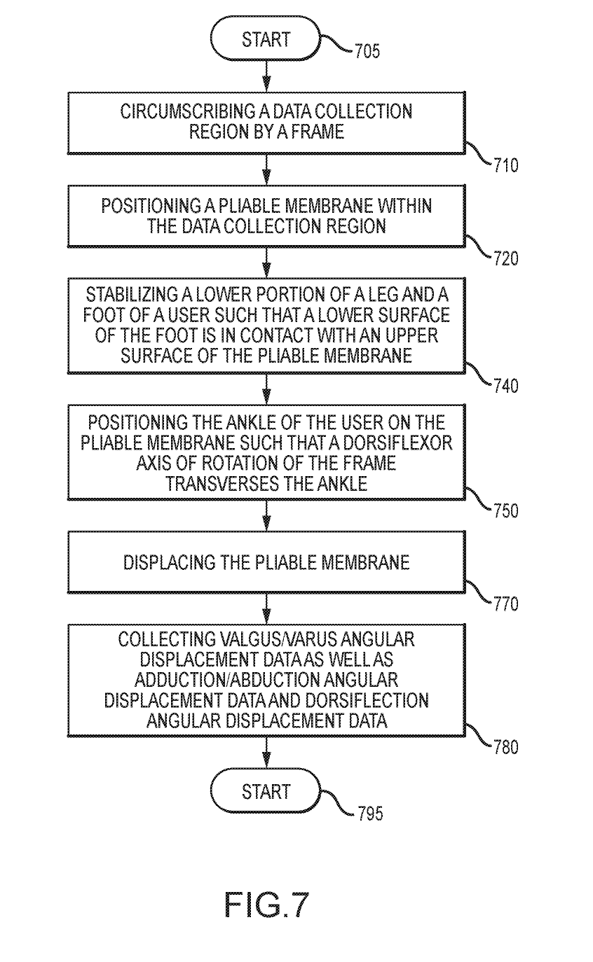

FIG. 7 presents a flowchart for one method embodiment for collecting foot-bed orthotic date using a foot-bed collection system according to the present invention. In the following description, it will be understood that each block of the flowchart illustrations, and combinations of blocks in the flowchart illustrations, can be implemented by a variety of means, including in some instances, a computer program. In the case of computer program instructions, these instructions may be loaded onto a computer or other programmable apparatus to produce a machine such that the instructions that execute on the computer or other programmable apparatus create means for implementing the functions specified in the flowchart block or blocks. These computer program instructions may also be stored in a computer-readable memory that can direct a computer or other programmable apparatus to function in a particular manner such that the instructions stored in the computer-readable memory produce an article of manufacture including instruction means that implement the function specified in the flowchart block or blocks. The computer program instructions may also be loaded onto a computer or other programmable apparatus to cause a series of operational steps to be performed in the computer or on the other programmable apparatus to produce a computer implemented process such that the instructions that execute on the computer or other programmable apparatus provide steps for implementing the functions specified in the flowchart block or blocks.

Accordingly, blocks of the flowchart illustrations support combinations of means for performing the specified functions and combinations of steps for performing the specified functions. It will also be understood that each block of the flowchart illustrations, and combinations of blocks in the flowchart illustrations, can be implemented by special purpose hardware-based computer systems that perform the specified functions or steps, or combinations of special purpose hardware and computer instructions.

The collection process begins 705 with forming a collection region. In one embodiment a data collection region is formed by circumscribing 710 an area with a ridged frame. A pliable membrane is positioned 720 within the collection region and fixedly attached to the edges of the frame. Included in the frame is a stabilizing arm comprising one or more supports that accept a leg of a user so as to position a foot on the pliable membrane.

A user stabilizes 740 a lower portion of their leg within the collection system such that the lower portion of the foot of the user is in contact with and displaces the pliable membrane placing the foot in a suspended yet loaded state. The ankle of the user is positioned 750 on the pliable membrane such that the lateral axis of rotation of the frame traverses tire ankle while the longitudinal axis of the frame bisects the foot from heel to toe. In doing so the ankle and knee are placed into a neutral state so that any deviation of the foot structure is revealed by the lower surface of the membrane.

With the foot and ankle in the proper position the foot displaces 770 the membrane toward a reference ground (suspension) force. The foot therefore experiences a force upon which to push against thus causing the banding tissues and bones to bad yet the leg and ankle are constrained to remain aligned. In doing so the pliable membrane will stretch downward and illustrate the natural neutral, alignment of the foot while under a suspended load. This natural alignment can be used to identify what sort of orthotic and with what angular dimensions are needed to interact with the foot so that the foot, ankle, knee and hip remain aligned when walking/running on a flat surface. These dimensions are collected 780 using a collection device ending 795 the process.

In a preferred embodiment, portions of the present invention can be implemented in software. Data collected from the system and devices described herein can be used to identify an optimal orthotic shape. Moreover, the same of the orthotic may differ based on the targeted applications. For instance, the same collection of foot geometric measurements can be used to craft unique orthotic inserts for a single user who skis, runs, hikes, stands at an assembly line, or any other of a wide variety of functionalities. Software programming code which embodies the present invention and associated algorithms is typically accessed by a microprocessor from long-term, persistent storage media of some type, such as a flash drive or hard drive. The software programming code may be embodied on any of a variety of known media for use with a data processing system, such as a diskette, hard drive, CD-ROM, or the like. The code may be distributed on such media, or may be distributed from the memory or storage of one computer system over a network of some type to other computer systems for use by such other systems. Alternatively, the programming code may be embodied in the memory of the device and accessed by a microprocessor using an internal bus. The techniques and methods for embodying software programming code in memory, on physical media, and/or distributing software code via networks are well known and will not be further discussed herein.

Generally, program modules include routines, programs, objects, components, data structures and the like that perform particular tasks or implement particular abstract data types. Moreover, those skilled in the art will appreciate that the invention can be practiced with other computer system configurations, including hand-held devices, multi-processor systems, microprocessor-based or programmable consumer electronics, network PCs, minicomputers, mainframe computers, and the like. The invention may also be practiced in distributed computing environments where tasks are performed by remote processing devices that are linked through a communications network. In a distributed computing environment, program modules may be located in both local and remote memory storage devices.

An exemplary system for implementing the invention includes a general purpose computing device such as the form of a conventional personal computer, a personal communication device or the like, including a processing unit, a system memory, and a system bus that connects various system components, including the system memory to the processing unit. The system bus may be any of several types of bus structures including a memory bus or memory controller, a peripheral bus, and a local bus using any of a variety of bus architectures. The system memory generally includes read-only memory (ROM) and random access memory (RAM). A basic input/output system (BIOS), containing the bask routines that help to transfer information between elements within the personal computer, such as during start-up, is stored in ROM. The personal computer may further include a hard disk drive for reading from and writing to a hard disk, a magnetic disk drive for reading from or writing to a removable magnetic disk. The hard disk drive and magnetic disk drive are connected to the system bus by a hard disk drive interface and a magnetic disk drive interlace, respectively. The drives and their associated computer-readable media, provide non-volatile storage of computer readable instructions, data structures, program modules and other data for the personal computer. Although the exemplary environment described herein employs a hard disk and a removable magnetic disk, it should be appreciated by those skilled in the art that other types of computer readable media which can store data that is accessible by a computer may also be used in the exemplary operating environment.

While there have been described above the principles of the present invention in conjunction with a system for foot geometry data collection, it is to be clearly understood that the foregoing description is made only by way of example and not as a limitation to the scope of the invention. Particularly, it is recognized that the teachings of the foregoing disclosure will suggest other modifications to those persons skilled in the relevant art. Such modifications may involve other features that are already known per se and which may be used instead, of or in addition to features already described herein. Although claims have been formulated in this application to particular combinations of features, it should be understood that the scope of the disclosure herein also includes any novel feature or any novel combination of features disclosed either explicitly or implicitly or any generalization or modification thereof which would be apparent to persons skilled in the relevant art, whether or not such relates to the same invention as presently claimed in any claim and whether or not it mitigates any or all of the same technical problems as confronted by the present invention. The Applicant hereby reserves foe right to formulate new elate to such features and/or combinations of such features during the prosecution of the present application or of any further application derived therefrom.

1. A system for collection of foot geometry data, comprising:
a frame circumscribing a data collection region and defining a collection plane;
a pliable membrane positioned within the data collection region and within the collection plane wherein the pliable membrane includes an upper surface and a lower surface;
stabilizing arm coupled to the frame and extending away from the collection plane so as to position a lower portion of a leg and a foot of the user such that a lower surface of the foot of the user is in contact with the upper surface of the pliable membrane; and
a data collection device positioned below the frame and in direct view of the lower surface of the pliable membrane configured to measure defection of the pliable membrane away from the collection plane.

2. The system for collection of foot geometry data according to claim 1, further comprising a lateral axis of rotation extending through the collection plane and through a juncture of the stabilizing arm and the frame and wherein the frame is configured to pivot about the lateral axis of rotation with respect to the stabilising arm.

3. The system for collection of foot geometry data according to claim 2, wherein the foot of the user includes an ankle and wherein the foot is positioned such that the lateral axis of rotation extends through the ankle.

4. The system for collection of foot geometry data according to claim 2, further comprising a vertical axis of rotation extending through the collection plane and perpendicular to the lateral axis of rotation and wherein the frame is configured to pivot about the vertical axis of rotation.

5. The system for collection of foot geometry data according to claim 4, wherein the data collection device includes a machine capable of executing instructions embodied as software and a plurality of software portions and wherein one of said software portions is configured to identify angular deviations of the foot from the lower surface of the pliable membrane as displaced by the foot as the foot, retains a neutral foot geometry.

6. Hie system for collection of foot geometry data according to claim 4, wherein the data collection device includes a machine capable of executing instructions embodied as software and a plurality of software portions and wherein one of said software portions is configured to identify a neutral foot geometry of the lower surface of the pliable membrane as displaced by the foot with respect a line parallel to the collection plane as the frame is displaced about the lateral axis.

7. The system for collection of foot geometry data according to claim 4, wherein the frame is configured to independently rotate about the lateral axis of rotation and the vertical axis of rotation.

8. The system for collection of foot geometry data according to claim 1, wherein the data collection device measures an angular displacement and a range of motion.

9. The system for collection of foot geometry data according to claim 1, wherein the data collection device measures a longitudinal angular displacement of the foot with respect to the collection plane and a range of motion.

10. The system for collection of foot geometry data according to claim 1, further comprising aperture guides configured to capture the foot of the user on the upper surface of the pliable membrane and maximize contact between the lower surface of the foot and the upper surface of the pliable membrane as the pliable membrane is displaced.

11. A method for collection foot geometry data, the method comprising:
circumscribing a data collection region by a frame defining a collection plane;
positioning a pliable membrane within the data collection region and within the collection plane wherein the pliable membrane includes an upper surface and a lower surface;
stabilizing a lower portion of a leg and a foot of a user by a stabilizing arm coupled to the fame such that a lower surface of the foot of the user is to contact with the upper surface of the pliable membrane and wherein an ankle of the user is positioned such that a lateral axis of rotation extends through the ankle and a juncture of the stabilizing arm and the frame
displacing the pliable membrane away torn the collection plane
collecting foot geometry data by a data collection device positioned below the frame and in direct, view of the lower surface of the pliable membrane wherein collecting includes measuring an angular deviation of the foot about the longitudinal axis with respect to the collection plane.

12. The method for collection foot geometry data according to claim 11, further comprising pivoting the stabilizing arm about foe lateral axis of rotation and responsive to pivoting the stabilizing arm collecting one or more displaced angular deviation measurements of the foot about the longitudinal axis with respect to the collection plane.

13. The method for collection foot geometry data according to claim 11, further comprising, extending a longitudinal axis of rotation through the collection plane and perpendicular to the lateral axis of rotation and pivoting the frame about the lateral axis of rotation with respect to the stabilizing arm to result in dorsiflexion of the foot.

14. The method for collection foot geometry data according to claim 13, wherein collecting includes identifying angular deviations of the lower surface of the pliable membrane as displaced by the foot as the foot retains a neutral configuration.

15. The method for collection foot geometry data according to claim 11, further comprising rotating the frame independently about the lateral axis of rotation and the vertical axis of rotation.

16. Hie method for collection foot geometry data according to claim 11, wherein collecting includes measuring, by the data collection device, an angular displacement the foot about the longitudinal axis.

17. The method for collection foot geometry data according to claim 11, wherein collecting includes measuring, by the data collection device, an angular displacement the foot about the longitudinal axis under a plurality of displacements of the ankle about the lateral axis.

18. The method for collection foot geometry data according to claim 11, wherein collecting includes measuring, by the data collection device, an angular collecting includes measuring, by the data collection device, an angular displacement the foot about the longitudinal axis under a plurality of displacements of the ankle about the vertical axis.

19. The method for collection foot geometry data according to claim 11, further comprising capturing the foot of the user on the upper surface of the pliable membrane by aperture guides to maximize contact between the lower surface of the foot and the upper surface of the pliable membrane as the pliable membrane is displaced.
